SEQUENCE LISTING (5 sequences)

SEQ ID NO: 1 - protein, 976 aa, Homo sapiens [sequence omitted]

SEQ ID NO: 2 - DNA, 30 bp, Homo sapiens
  Primer
aagaagagatggtacctcgaggggtgaccc

SEQ ID NO: 3 - DNA, 33 bp, Homo sapiens
  Primer
ctgcttcgcggccgcgttaactcttctcaacca

SEQ ID NO: 4 - DNA, 20 bp, Homo sapiens
  Primer
agctcgtttagtgaaccgtc

SEQ ID NO: 5 - DNA, 20 bp, Homo sapiens
  Primer
gtcagacaaaatgatgcaac

(19) United States
(12) Patent Application Publication
(10) Pub. No.: US 2004/0266771 A1
(43) Pub. Date: Dec. 30, 2004

(54) Use of tyrosine kinase inhibitors for treating bone loss

(76) Inventors: Alain Moussy, Paris (FR); Jean-Pierre Kinet, Lexington, MA (US)

Correspondence Address: SUGHRUE MION, PLLC, 2100 PENNSYLVANIA AVENUE, N.W., SUITE 800, WASHINGTON, DC, 20037, US

(21) Appl. No.: 10/482,036

(22) PCT Filed: Jun. 28, 2002
(86) PCT No.: PCT/IB02/03288

Related U.S. Application Data
(60) Provisional application No. 60/301,411, filed on Jun. 29, 2001.

(51) Int. Cl. A61K 31/50; A61K 31/495; A61K 31/519; A61K 31/517; A61K 31/505; A61K 31/47

(52) U.S. Cl. 514/247; 514/248; 514/265.1; 514/266.4; 514/275; 514/312; 514/414

(57) ABSTRACT

The present invention relates to a method for treating bone loss such as osteoporsis comprising administering a tyrosine kinase inhibitor to a human in need of such treatment, more particularly a non-toxic, selective and potent c-kit inhibitor. Preferbly, said inhibitor is unable to promote death of IL-3 dependent cells cultured in presence of IL-3.

The present invention relates to a method for treating bone loss such as osteoporosis comprising administering a tyrosine kinase inhibitor to a human in need of such treatment, more particularly a non-toxic, selective and potent c-kit inhibitor. Preferably, said inhibitor is unable to promote death of IL-3 dependent cells cultured in presence of IL-3.

Bone is a living and growing tissue mostly made of a collagen framework and calcium phosphate, a mineral that strengthens the framework. Both collagen and calcium allow bones to withstand mechanical stress. During the lifetime, bones become larger, heavier, and denser until a maximum is reached around age 30. Then, the balance between bone resorption and growth starts to invert and rapid bone loss in observed in the first few years after menopause but persists into the postmenopausal years. Osteoporosis develops when the balance between resorption and growth turns suddenly and significantly in favor of boss loss. A review of this disease can be found in Raisz et al, 2000 Epidemiology and pathogenesis of osteoporosis. Clin Cornerstone, 2(6):1-10.

Osteoporosis concerns about 30 million Americans, 80% of whom are women. In addition, it is estimated that one out of every two women and one in eight men over 50 will have an osteoporosis-related fracture in their lifetime. Osteoporosis is responsible for more than 1.5 million fractures annually in the USA alone and the cost relating to osteoporosis is about $14 billion each year.

Current methods for treating or preventing osteoporosis include administration of estrogen, calcitonin, alendronate, raloxifene, and risedronate. Estrogen replacement therapy has been shown to reduce bone loss, increase bone density but it can increase a woman's risk of developing cancer of the uterine lining. Raloxifene is a selective estrogen receptor modulators that appear to prevent bone loss but side effects such as hot flashes and deep vein thrombosis have been observed. Alendronate belongs to the class of drugs called bisphosphonates and was demonstrated to reduces bone loss, increases bone density but abdominal or musculoskeletal pain, nausea, heartburn, or irritation of the esophagus have also been observed. Calcitonin is a naturally occurring non-sex hormone involved in calcium regulation and bone metabolism. In women who are at least 5 years beyond menopause, calcitonin slows bone loss and relieves the pain associated with bone fractures. However, injectable calcitonin may cause an allergic reaction and unpleasant side effects including flushing of the face and hands, urinary frequency, nausea, and skin rash. Treatments used for bone loss in men also include vitamin and mineral supplementation with calcium and vitamin D but this has limited effectiveness in treating advanced disease.

Therefore, there is a need for alternative treatments of bone loss that would be more effective on the long term in regards to the above mentioned observations and which would be well tolerated especially in respect to repeated administration.

In connection with the invention, we found that an increased parathyroid hormone secretion, certain cytokines, and other bone-resorbing mediators can stimulate bone resorption. Low serum calcium levels promote parathyroid hormone secretion, and estrogen deficiency is associated with a rise in cytokine production and activity. An abnormal proliferation of mast cells may also release cytokines, heparin, and other mediators of bone resorption. Of interest, mast cell proliferation has been reported in disorders of abnormal bone remodeling. For example, severe osteoporosis due to systemic mast cell disease has been observed by Lehmann T et al, Br J Rheumatol. September 1996;35(9):898-900. In fact, osteoporosis can be a symptoms in some cases of mastocytosis, Johansson C. et al, 1996, Age Ageing. January;25(1):1-7 and Delsignore J L et al, 1996. Iowa Orthop J.;16:126-34.

Quantification of the number of mast cells in undecalcified section of iliac crest bone from untreated women with postmenopausal osteoporosis contrasted the findings to values from normal women and normal men. The mean number of marrow mast cells is greater in normal women than men. Compared to the normal women, osteoporotic women had a greater number of mast cells in the marrow. Here, these findings confirm the association between increased numbers of mast cells and postmenopausal osteoporosis.

Therapeutic strategies aiming at blocking the activation and the survival of mast cells, for instance through inhibition of c-kit or c-kit signaling might thus be beneficial and could help to decrease the manifestations of the disease.

Mast cells (MC) are tissue elements derived from a particular subset of hematopoietic stem cells that express CD34, c-kit and CD13 antigens (Kirshenbaum et al, Blood. 94: 2333-2342, 1999 and Ishizaka et al, Curr Opin Immunol. 5: 937-43, 1993). Immature MC progenitors circulate in the bloodstream and differentiate in tissues. These differentiation and proliferation processes are under the influence of cytokines, one of utmost importance being Stein Cell Factor (SCF), also termed Kit ligand (KL), Steel factor (SL) or Mast Cell Growth Factor (MCGF). SCF receptor is encoded by the protooncogene c-kit, that belongs to type III receptor tyrosine kinase subfamily (Boissan and Arock, J Leukoc Biol. 67: 135-48, 2000). This receptor is also expressed on others hematopoietic or non hematopoietic cells. Ligation of c-kit receptor by SCF induces its dimerization followed by its transphosphorylation, leading to the recruitment and activation of various intracytoplasmic substrates. These activated substrates induce multiple intracellular signaling pathways responsible for cell proliferation and activation (Boissan and Arock, 2000). Mast cells are characterized by their heterogeneity, not only regarding tissue location and structure but also at the functional and histochemical levels (Aldenborg and Enerback., Histochem. J. 26: 587-96, 1994; Bradding et al, J Immunol. 155: 297-307, 1995; Irani et al, J Immunol. 147: 247-53, 1991; Miller et al, Curr Opin Immunol. 1: 637-42, 1989 and Welle et al, J Leukoc Biol. 61: 233-45, 1997).

In connection with the invention, it is proposed that mast cells play a crucial role in the pathogenesis of bone loss, such as osteoporosis, including post menopausal osteoporosis, senile osteoporosis, and glucocorticoid-induced osteoporosis, osteitis fibrosa cystica, renal osteodystrophy, osteosclerosis, osteopenia, osteomalacia, fibrogenesis-imperfecta ossium, and Paget's Disease in that they produce a large variety of mediators categorized here into three groups:

preformed granule-associated mediators (histamine, proteoglycans, and neutral proteases),

lipid-derived mediators (prostaglandins, thromboxanes and leucotrienes),

and various cytokines (IL-1, IL-2, IL-3, IL-4, IL-5, IL-6, IL-8, TNF-a, GM-CSF, MIP-1a, MIP-1b and IFN-γ).

Then, liberation by activated mast cells of mediators (TNF-a, leucotrienes, prostaglandines etc . . . ) can induce local inflammation and activation of cell apoptosis in bones. In addition, mast cells activate T cells and macrophages, which further contributes to this inflammation and destruction process.

Therefore, the invention proposes to use c-kit specific kinase inhibitors to inhibit mast cell proliferation, survival and activation. A new route for treating bone loss is provided, which consists of destroying mast cells playing a role in the pathogenesis of these disorders. It has been found that tyrosine kinase inhibitors and more particularly c-kit inhibitors are especially suited to reach this goal.

### DESCRIPTION

The present invention relates to a method for treating bone loss comprising administering a tyrosine kinase inhibitor to a mammal in need of such treatment.

Tyrosine kinase inhibitors are selected for example from bis monocyclic, bicyclic or heterocyclic aryl compounds (WO 92/20642), vinylene-azaindole derivatives (WO 94/14808) and 1-cyclopropyl-4-pyridyl-quinolones (U.S. Pat. No. 5,330,992). Styryl compounds (U.S. Pat. No. 5.217,999), styryl-substituted pyridyl compounds (U.S. Pat. No. 5,302,606), seleoindoles and selenides (WO 94/03427), tricyclic polyhydroxylic compounds (WO 92/21660) and benzylphosphionic acid compounds (WO 91/15495), pyrimidine derivatives (U.S. Pat. No. 5,521,184 and WO 99/03854), indolinone derivatives and pyrrol-substituted indolinones (U.S. Pat. No. 5,792,783, EP 934 931. U.S. Pat. No. 5,834,504, U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940) and aryl and heteroaryl quinazoline (U.S. Pat. No. 5,721,237, U.S. Pat. No. 5,714.493, US 5,710,158 and WO 95/15758).

Preferably, said tyrosine kinase inhibitors are unable to promote death of IL-3 dependent cells cultured in presence of IL-3.

In another embodiment, the invention is directed to a method for treating bone loss comprising administering a c-kit inhibitor to a mammal in need of such treatment. Preferably, said c-kit inhibitor is a non-toxic, selective and potent c-kit inhibitor. Such inhibitors can be selected from the group consisting of indolinones, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

Among preferred compounds, it is of interest to focus on pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834.504), U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772.295 and U.S. Pat. No. 4,343,940), 4-amino-substituted quinazolines (U.S. Pat. No. 3,470,182), 4-thienyl-2-(1H)-quinazolones, 6,7-dialkoxyquinazolines (U.S. Pat. No. 3,800,039), aryl and heteroaryl quinazoline (U.S. Pat. No. 5,721,237, U.S. Pat. No. 5,714,493, U.S. Pat. No. 5,710,158 and WO 95/15758), 4-anilinoquinazoline compounds (U.S. Pat. No. 4,464,375), and 4-thienyl-2-(1H)-quinazolones (U.S. Pat. No. 3,551,427).

So, preferably, the invention relates to a method for treating bone loss comprising administering a non toxic, potent and selective c-kit inhibitor. Such inhibitor can be selected from pyrimidine derivatives, more particularly N-phenyl-2-pyrimidine-amine derivatives of formula I:

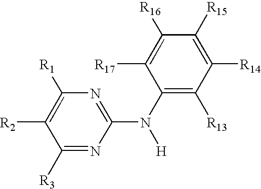

wherein the R1, R2, R3, R13 to R17 groups have the meanings depicted in EP 564 409 B1 incorporated herein in the description.

Preferably, the N-phenyl-2-pyrimidine-amine derivative is selected from the compounds corresponding to formula II:

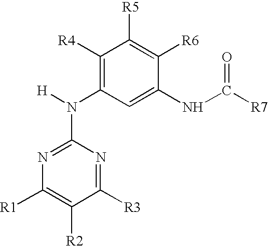

Wherein R1, R2 and R3 are independently chosen from H, F, Cl, Br, I, a C1-C5 alkyl or a cyclic or heterocyclic group, especially a pyridyl group;

R4, R5 and R6 are independently chosen from H, F, Cl, Br, I, a C1-C5 alkyl, especially a methyl group;

and R7 is a phenyl group bearing at least one substituent, which in turn possesses at least one basic site, such as an amino function.

Preferably, R7 is the following group

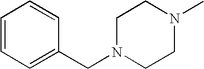

Among these compounds, the preferred are defined as follows:

R1 is a heterocyclic group, especially a pyridyl group,

R2 and R3 are H,

R4 is a C1-C3 alkyl, especially a methyl group,

R5 and R6 are H,

and R7 is a phenyl group bearing at least one substituent, which in turn possesses at least one basic site, such as an amino function, for example the group:

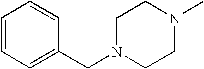

Therefore, in a preferred embodiment, the invention relates to a method for treating bone loss comprising the administration of an effective amount of the compound known in the art as CGP57148B:

4-(4-méhylpipérazine-1-ylméthyl)-N-[4-méthyl-3-(4-pyridine-3-yl)pyrimidine-2ylamino)phényl]-benzamide corresponding to the following formula:

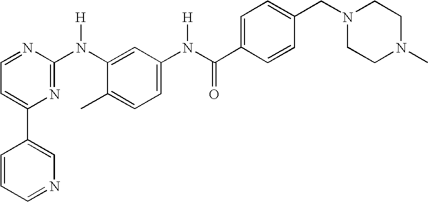

The preparation of this compound is described in example 21 of EP 564 409 and the β-form, which is particularly useful is described in WO 99/03854.

Alternatively, the c-kit inhibitor can be selected from

indolinone derivatives, more particularly pyrrol-substituted indolinones,

monocyclic, bicyclic aryl and heteroaryl compounds quinazoline derivatives,

and quinaxolines, such as 2-phenyl-quinaxoline derivatives, for example 2-phenyl-6,7-dimethoxy quinaxoline.

In a preferred aspect, the invention contemplated the method mentioned above, wherein said c-kit inhibitor is unable to promote death of IL-3 dependent cells cultured in presence of IL-3.

The expression “bone loss” refers herein to a disease selected from osteoporosis, including post menopausal osteoporosis, senile osteoporosis, and glucocorticoid-induced osteoporosis, osteitis fibrosa cystica, renal osteodystrophy, osteosclerosis, osteopenia, osteomalacia, fibrogenesis-imperfecta ossium, and Paget's Disease.

In a further embodiment, c-kit inhibitors as mentioned above are inhibitors of activated c-kit. In frame with the invention the expression “activated c-kit” means a constitutively activated-mutant c-kit including at least one mutation selected from point mutations, deletions, insertions, but also modifications and alterations of the natural c-kit sequence (SEQ ID N°1). Such mutations, deletions, insertions, modifications and alterations can occur in the transphosphorylase domain, in the juxtamembrane domain as well as in any domain directly or indirectly responsible for c-kit activity. The expression “activated c-kit” also means herein SCF-activated c-kit. Preferred and optimal SCF concentrations for activating c-kit are comprised between 5.10⁻⁷ M and 5.10⁻⁶ M, preferably around 2.10⁻⁶ M. In a preferred embodiment, the activated-mutant c-kit in step a) has at least one mutation proximal to Y823, more particularly between amino acids 800 to 850 of SEQ ID No1 involved in c-kit autophosphorylation, notably the D816V, D816Y, D816F and D820G mutants. In another preferred embodiment, the activated-mutant c-kit in step a) has a deletion in the juxtamembrane domain of c-kit. Such a deletion is for example between codon 573 and 579 called c-kit d(573-579). The point mutation V559G proximal to the juxtamembrane domain c-kit is also of interest.

In this regard, the invention contemplates a method for treating bone loss comprising administering to a mammal in need of such treatment a compound that is a selective, potent and non toxic inhibitor of activated c-kit obtainable by a screening method which comprises:

a) bringing into contact (i) activated c-kit and (ii) at least one compound to be tested; under conditions allowing the components (i) and (ii) to form a complex,

b) selecting compounds that inhibit activated c-kit,

c) testing and selecting a subset of compounds identified in step b), which are unable to promote death of IL-3 dependent cells cultured in presence of IL-3.

This screening method can further comprise the step consisting of testing and selecting a subset of compounds identified in step b) that are inhibitors of mutant activated c-kit (for example in the transphosphorylase domain), which are also capable of inhibiting SCF-activated c-kit wild.

Alternatively, in step a) activated c-kit is SCF-activated c-kit wild.

A best mode for practicing this method consists of testing putative inhibitors at a concentration above 10 μM in step a). Relevant concentrations are for example 10, 15, 20, 25, 30, 35 or 40 μM.

In step c), IL-3 is preferably present in the culture media of IL-3 dependent cells at a concentration comprised between 0.5 and 10 ng/ml, preferably between 1 to 5 ng/ml.

Examples of IL-3 dependent cells include but are not limited to:

cell lines naturally expressing and depending on c-kit for growth and survival. Among such cells, human mast cell lines can be established using the following procedures normal human mast cells can be infected by retroviral vectors containing sequences coding for a mutant c-kit comprising the c-kit signal peptide and a TAG sequence allowing to differentiate mutant c-kits from c-kit wild expressed in hematopoetic cells by means of antibodies.

This technique is advantageous because it does not induce cellular mortality and the genetic transfer is stable and gives satisfactory yields (around 20%). Pure normal human mast cells can be routinely obtained by culturing precursor cells originating from blood obtained from human umbilical vein. In this regard, heparinated blood from umbilical vein is centrifuged on a Ficoll gradient so as to isolate mononucleated cells from other blood components. CD34+ precursor cells are then purified from the isolated cells mentioned above using the immunomagnetic selection system MACS (Miltenyi biotech). CD34+ cells are then cultured at 37° C. in 5% CO₂ atmosphere at a concentration of 10⁵ cells per ml in the medium MCCM (α-MEM supplemented with L-glutamine, penicillin, streptomycin, 5 10⁻⁵ M β-mercaptoethanol. 20% veal fcetal serum, 1% bovine albumin serum and 100 ng/ml recombinant human SCF. The medium is changed every 5 to 7 days. The percentage of mast cells present in the culture is assessed each week, using May-Grünwal Giemsa or Toluidine blue coloration. Anti-tryptase antibodies can also be used to detect mast cells in culture. After 10 weeks of culture, a pure cellular population of mast cells (>98%) is obtained.

It is possible using standard procedures to prepare vectors expressing c-kit for transfecting the cell lines established as mentioned above. The cDNA of human c-kit has been described in Yarden et al., (1987) EMBO J.6 (11), 3341-3351. The coding part of c-kit (3000 bp) can be amplified by PCR and cloned, using the following oligonucleotides:

| | |
|---|---|
| 5′AAGAAGAGATGGTACCTCGAGGGGTGACCC3′ | (SEQ ID No2) |
| sens | |
| 5′CTGCTTCGCGGCCGCGTTAACTCTTCTCAACCA3′ | (SEQ ID No3) |
| antisens | |

The PCR products, digested with NotI and XhoI, has been inserted using T4 ligase in the pFlag-CMV vector (SIGMA), which vector is digested with NotI and XhoI and dephosphorylated using CIP (Biolabs). The pFlag-CMV-c-kit is used to transform bacterial clone XLI-blue. The transformation of clones is verified using the following primers:

| | | |
|---|---|---|
| 5′AGCTCGTTTAGTGAACCGTC3′ | (SEQ ID No4) | sens, |
| 5′GTCAGACAAAATGATGCAAC3′ | (SEQ ID No5) | antisens. |

Directed mutagenesis is performed using relevant cassettes is performed with routine and common procedure known in the art.

The vector Migr-1 (ABC) can be used as a basis for constructing retroviral vectors used for transfecting mature mast cells. This vector is advantageous because it contains the sequence coding for GFP at the 3′ and of an IRES. These features allow to select cells infected by the retrovirus using direct analysis with a fluorocytometer. As mentioned above, the N-terminal sequence of c-kit c-DNA can be modified so as to introduce a Flag sequence that will be useful to discriminating heterogeneous from endogenous c-kit.

Other IL-3 dependent cell lines that can be used include but are not limited to:

BaF3 mouse cells expressing wild-type or mutated form of c-kit (in the juxtamembrane and in the catalytic sites) are described in Kitayama et al, (1996), Blood 88, 995-1004 and Tsujimura et al, (1999), Blood 93, 1319-1329.

IC-2 mouse cells expressing either c-kit^{WT} or c-kit^{D84Y} are presented in Piao et al, (1996), Proc. Natl. Acad. Sci. USA 93, 14665-14669.

IL-3 independent cell lines are:

HMC-1, a factor-independent cell line derived from a patient with mast cell leukemia, expresses a juxtamembrane mutant c-kit polypeptide that has constitutive kinase activity (Furitsu T et al, J Clin Invest. 1993;92: 1736-1744; Butterfield et al, Establishment of an immature mast cell line from a patient with mast cell leukemia. Leuk Res. 1988;12:345-355 and Nagataet al, Proc Natl Acad Sci USA. 1995.92:10560-10564).

P815 cell line (mastocytoma naturally expressing c-kit mutation at the 814 position) has been described in Tsujimura et al. (1994), Blood 83, 2619-2626.

The extent to which component (ii) inhibits activated c-kit can be measured in vitro or in vivo. In case it is measured in vivo, cell lines expressing an activated-mutant c-kit, which has at least one mutation proximal to Y823, more particularly between amino acids 800 to 850 of SEQ ID No1 involved in c-kit autophosphorylation, notably the D816V, D816Y, D816F and D820G mutants, are preferred.

Example of cell lines expressing an activated-mutant c-kit are as mentioned.

In another preferred embodiment, the method further comprises the step consisting of testing and selecting compounds capable of inhibiting c-kit wild at concentration below 1 μM. This can be measured in vitro or in vivo.

In vivo testing may comprise measuring the ability of the tyrosine kinase inhibitors to alleviate osteoporosis symptoms in transgenic mouse model of osteoporosis. For example, a transgenic mouse that lacks endogenous SPARC expression can be useful in this regard (U.S. Pat. No. 6,239,326).

Therefore, compounds are identified and selected according to the method described above are potent, selective and non-toxic c-kit wild inhibitors.

Alternatively, the screening method as defined above can be practiced in vitro. In this regard, the inhibition of mutant-activated c-kit and/or c-kit wild can be measured using standard biochemical techniques such as immunoprecipitation and western blot. Preferably, the amount of c-kit phosphorylation is measured.

In a still further embodiment, the invention contemplates a method for treating bone loss as depicted above wherein the screening comprises:

a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell deaths

b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,

c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

Here, the extent of cell death can be measured by 3H thymidine incorporation, the trypan blue exclusion method or flow cytometry with propidium iodide. These are common techniques routinely practiced in the art.

The method according to the invention includes preventing and/or treating bone loss in human.

Therefore, the invention embraces the use of the compounds defined above to manufacture a medicament for treating bone loss such as osteoporosis, including post menopausal osteoporosis, senile osteoporosis, and glucocorticoid-induced osteoporosis, osteitis fibrosa cystica, renal osteodystrophy, osteosclerosis, osteopenia, osteomalacia, fibrogenesis-imperfecta ossium, and Paget's Disease.

The pharmaceutical compositions utilized in this invention may be administered by any number of routes including, but not limited to oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Reminigton's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical compositions suitable for use in the invention include compositions wherein c-kit inhibitors are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therpeutic effects is the therapeutic index, and it can be expressed as the ratio, LD50/ED50. Pharmaceutical compositions which exhibit large therapeutic indices are preferred. As mentioned above, a tyrosine kinase inhibitor and more particularly a c-kit inhibitor according to the invention is unable to promote death of IL-3 dependent cells cultured in presence IL-3.

The invention also concerns a product comprising a tyrosine kinase inhibitor as defined above and at least one compound selected from estrogen, calcitonin, alendronate, raloxifene, risedronate, vitamin D and calcium for a separate, simultaneous or concomitant use for treating bone loss.

1. A method for treating bone loss comprising administering a tyrosine kinase inhibitor to a mammal in need of such treatment.

2. A method according to claim 1, wherein said tyrosine kinase inhibitor is unable to promote death of IL-3 dependent cells cultured in presence of IL-3.

3. A method for treating bone loss comprising administering a c-kit inhibitor to a mammal in need of such treatment.

4. A method according to claim 3, wherein said c-kit inhibitor is a non-toxic, selective and potent c-kit inhibitor.

5. A method according to claim 4, wherein said inhibitor is selected from the group consisting of indolinones, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

6. A method according to claim 4, wherein said inhibitor is selected from the group consisting of:
pyrimidine derivatives, more particularly N-phenyl-2-pyrimidine-amine derivatives
indolinone derivatives, more particularly pyrrol-substituted indolinones,
monocyclic, bicyclic aryl and heteroaryl compounds,
and quinazoline derivatives.

7. A method according to one of claims 3 to 6, wherein said c-kit inhibitor is selected from compounds of formula II: 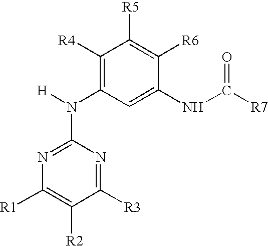
Wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function.

8. A method according to one of claims 3 to 6, wherein said c-kit inhibitor is the 4-(4-méhylpipérazine-1-ylméthyl)-N-(4-méthyl-3-(4-pyridine-3-yl)pyrimidine-2ylamino)phényl]-benzamide.

9. A method according to one of claims 3 to 8, wherein said c-kit inhibitor is unable to promote death of IL-3 dependent cells cultured in presence of IL-3.

10. A method according to one of claims 3 to 9, wherein said c-kit inhibitor is an inhibitor of activated c-kit.

11. A method according to claim 10, wherein said activated c-kit inhibitor is capable of inhibiting SCF-activated c-kit.

12. A method according to claim 10, wherein said inhibitor is capable of inhibiting constitutively activated-mutant c-kit.

13. A method for treating bone loss comprising administering to a mammal in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

14. A method according to claim 13, wherein the screening method further comprises the step consisting of testing and selecting a subset of compounds identified in step b) that are inhibitors of mutant activated c-kit, which are also capable of inhibiting SCF-activated c-kit wild.

15. A method according to claim 13, wherein activated c-kit is SCF-activated c-kit wild in step a).

16. A method according to one of claims 13 to 15 wherein putative inhibitors are tested at a concentration above 10 μM in step a).

17. A method according to one of claims 13 to 16 wherein IL-3 is preferably present in the culture media of IL-3 dependent cells at a concentration comprised between 0.5 and 10 ng/ml, preferably between 1 to 5 ng/ml.

18. A method according to claim 17, wherein IL-3 dependent cells are selected from the group consisting of mast cells, transfected mast cells. BaF3, and IC-2.

19. A method according to one of claims 13 to 18, wherein the extent to which component (ii) inhibits activated c-kit is measured in vitro or in vivo.

20. A method according to one of claims 13 to 19, further comprising the step consisting of testing and selecting compounds capable of inhibiting c-kit wild at concentration below 1 μM.

21. A method according to claim 20, wherein the testing is performed in vitro or in vivo.

22. A method according to one of claims 13 to 21, wherein the inhibition of mutant-activated c-kit and/or c-kit wild is measured using standard biochemical techniques such as immunoprecipitation and western blot.

23. A method according to one of claims 13 to 21, wherein the amount of c-kit phosphorylation is measured.

24. A method according to one of claims 13 to 23, wherein identified and selected compounds are potent, selective and non-toxic c-kit wild inhibitors.

25. A method for treating bone loss comprising administering to a mammal in need of such treatment a c-kit inhibitor obtainable by a screening method comprising
a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,
b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,
c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

26. A method according to claim 25, wherein the extent of cell death is measured by 3H thymidine incorporation, the trypan blue exclusion method or flow cytometry with propidium iodide.

27. A method according to one of claims 1 to 26 for preventing and/or treating bone loss in human.

28. A method according to one of claims 1 to 26 for preventing and/or treating bone loss such as osteoporosis, including post menopausal osteoporosis, senile osteoporosis, and glucocorticoid-induced osteoporosis, osteitis fibrosa cystica, renal osteodystrophy, osteosclerosis, osteopenia, osteomalacia, fibrogenesis-imperfecta ossium, and Paget's Disease.

29. Use of a c-kit inhibitor to manufacture a medicament for treating bone loss.

30. A composition suitable for oral administration comprising a tyrosine kinase inhibitor, more particularly a c-kit inhibitor for the treatment of bone loss such as osteoporosis, including post menopausal osteoporosis, senile osteoporosis, and glucocorticoid-induced osteoporosis, osteitis fibrosa cystica, renal osteodystrophy, osteosclerosis, osteopenia, osteomalacia, fibrogenesis-imperfecta ossium, and Paget's Disease.

31. A composition suitable for topical, intranasal, intravenous, intramuscular, intra-arterial, intramedullary, intrathecal, intraventricular, transdermal, subcutaneous, intraperitoneal, enteral, sublingual, or rectal administration comprising a tyrosine kinase inhibitor, more particularly a c-kit inhibitor for the treatment of bone loss such as osteoporosis, including post menopausal osteoporosis, senile osteoporosis, and glucocorticoid-induced osteoporosis, osteitis fibrosa cystica, renal osteodystrophy, osteosclerosis, osteopenia, osteomalacia, fibrogenesis-imperfecta ossium and Paget's Disease.
